(19) United States
(12) Patent Application Publication
(10) Pub. No.: US 2022/0273930 A1
(43) Pub. Date: Sep. 1, 2022

(54) INTELLIGENT MEDICAL FLUID CONDUCTION AND TRANSFER SYSTEM, AND COMPONENTS THEREFOR

(71) Applicant: ConnCons GmbH, Radebeul (DE)

(72) Inventors: Christoph GOMMEL, Radebeul (DE); Jochen HAMPE, Dresden (DE); Carsten SCHURIG, Radebeul (DE)

(21) Appl. No.: 17/632,089

(22) PCT Filed: Jul. 30, 2020
(86) PCT No.: PCT/EP2020/071596

§ 371 Date: Feb. 1, 2022

(30) Foreign Application Priority Data
Aug. 2, 2019 (DE) 10 2019 120 926.9

(51) Int. Cl. A61M 39/10; G16H 20/17; G16H 40/63; A61M 60/37; A61M 60/109

(52) U.S. Cl. CPC A61M 39/10; G16H 20/17; G16H 40/63; A61M 60/37; A61M 60/109; A61M 2039/1005; A61M 2039/1022; A61M 2039/1044

(57) ABSTRACT

A connecting piece having a transfer apparatus for data transfer and/or for transferring electrical energy with the counter connecting piece. A connecting piece of this type is preferably used in an intelligent medical fluid conduction and transfer system, in which various components are interconnected by means of the connecting pieces not only fluidically but also for the purpose of transferring energy and/or data with each other, such that, for example, information regarding the medication can be exchanged or validated.

### FIELD OF APPLICATION AND PRIOR ART

The invention relates to a medical fluid conduction and transfer system and to components which can be used in such a system.

A medical fluid conduction and transfer system is a system for guiding fluids in connection with the medical treatment of a person or an animal. Such a system can be, for example, an infusion system or a transfusion system or a dialysis system or an extracorporeal gas-exchange system or an extracorporeal circulatory support system or a heart-lung machine or a feeding system or a drainage system for body fluids, such as urine, wound secretion or intestinal contents, or a ventilation system.

A fluid conduction and transfer system of the type in question and of the invention usually has at least one fluid source for providing a medically relevant fluid or fluid mixture, an access unit for administering the medically relevant fluid or fluid mixture in a patient, and a connecting conductor system which connects the fluid source and the access unit and is designed for conduction and transfer of the medically relevant fluid mixture from the fluid source to the patient, the connecting conductor system usually having at least one regulating transfer element.

A fluid or fluid mixture in the context of the invention can be gaseous, for example respiratory air, ambient air enriched with anesthetic gas, or oxygen; or it can be liquid, for example an infusion solution, a drug, urine, blood, a dialysate or precursors of dialysates.

A fluid source is understood to mean a storage unit or a media supply for the fluid or fluid mixture, for example wall connections for oxygen or natural sources.

An access unit can, for example, be formed by a needle or a temporary vascular line such as a central venous catheter or an arterial line, which is introduced into a vein or artery of the patient. An access unit can also be formed by a port, i.e., a subcutaneously implanted catheter system which can be punctured from the outside and offers permanent access to the arterial or venous vascular system or to certain body cavities.

The connecting conductor system in the context of the invention combines all the components of the fluid conduction and transfer system that are necessary for conducting and transferring or distributing the medically relevant fluid or fluid mixture. A regulating transfer element such as, for example, a three-way valve, a valve bank or a filter element is part of the connecting conductor system in the context of the invention. A transfer element is understood here to mean an element which serves to simultaneously forward or transfer medically relevant fluids or fluid mixtures from multiple fluid sources into an access unit on the patient, which fluid sources are connected to the transfer element via, in each case, a connecting conductor of the connecting conductor system.

Practically every inpatient receives administration of drugs, chemotherapy, infusion fluids or feeding solutions via an access unit, i.e., peripheral intravenous (IV) indwelling cannulas, temporary central indwelling catheters or permanently implanted vascular lines (so-called “ports”). These are also called IV therapies. Almost all of these therapies are already being carried out using pumps, for example infusion pumps in infusion systems, which pumps are usually programmable offline, i.e., the pump parameters are preset according to their use on the patient and associated with the patient.

In the case of patients who are in intensive care units, under anesthesia or suffering from a severe disease, a multiplicity of IV therapies are often carried out at the same time via different IV lines. The number can be up to 30. In these situations, multiple parallel intravenous lines are necessary for controlled administration of life-sustaining and mutually incompatible drugs in a precisely controllable manner. The correct administration and documentation of these therapies is of huge relevance to patient safety and the medico-legal situation of the treating nurses, physicians and institutions. To carry out these therapies, billions of disposable items are manufactured annually.

The infusion and connection systems of today were conceived at the end of the 19th century and are geared especially toward good functionality purely under the action of gravity. The movement of the infusion liquid can, however, also be realized by means of an infusion pump or by active liquid outflow from the fluid source, for example from a storage container for the infusion liquid. On the path to the access unit, for example an IV line on the patient, one or more regulating transfer elements can be formed. The Luer system, which is virtually exclusively used internationally for IV therapies, dates back to the instrument maker Hermann Wülfing Luer (year of death: 1883). The combination of the Luer connection with a rotation lock (Luer lock, as a development of the Luer slip plug connection) was first described in 1952. The system is internationally standardized in the ISO standard ISO 80369-7. In its current form, the components of the infusion system or of a fluid conduction and transfer system are manually plugged or screwed together and manually checked for physically correct connection thereof.

The infusion liquids are transferred from a storage container to an IV line on the patient via a connecting conductor system, which is formed by the infusion lines or tubes in the case of an infusion system, the infusion pump and the regulating transfer elements.

For a functional and medically effective fluid conduction and transfer system, what is essential is correct functioning and inter-connection of all the components. In the case of an infusion system, it must be ensured, for example, that the correct drug is administered via an appropriate infusion pump at the correct infusion rate, and it is necessary to ensure a connection right into the patient that is continuous and in line with compatibility and safety criteria, so that, for example, incompatible drugs and infusion solutions cannot be jointly administered. With the hitherto known infusion systems, correct connection is checked manually by the physician or a nurse.

For the safe functioning of a medical fluid conduction system, it is essential to observe hygiene standards to protect the patient. This concerns especially the correct use of filters, the avoidance of unnecessary reconnections and adherence to the change intervals. Hitherto known fluid conduction systems do not allow any checking or monitoring of these parameters. Instead, checking and documentation are done manually by the physician or a nurse.

Medical fluid conduction systems can pose a significant risk to fellow patients, relatives or nursing staff. For example, this is the case when the fluid conduction system has been filled with highly toxic substances, such as in the case of chemotherapy, and said substances escape as a result of leakage or operating errors. Hitherto existing systems are not able to detect the escape of chemotherapeutic.

Furthermore, documentation of therapy, an integrity check, a plausibility test or feedback on status and problems in the context of, for example, infusion therapy is not possible with the fluid conduction and transfer systems used to date. These constraints mean that patient safety suffers, and a large amount of manual documentation is required. Electronic interlinkage of, for example, infusion therapy with the other procedures for therapy is not possible.

### OBJECT AND ACHIEVEMENT

It is an object of the invention to provide a fluid conduction and transfer system which does not have the stated deficiencies or only has them to a lesser extent and which furthermore preferably meets the following functional requirements: 1. Ideally, the fluid conduction and transfer system should be able to independently validate its integrity from the fluid source, i.e., from the drug container, infusion bag, drug bottle or the like, up to the patient, to detect misconnections and drug incompatibilities and to provide validated documentation of the therapy. Validation is to be understood here to mean that a drug has not only passed through, for example, the infusion pump, but has also entered an IV catheter. 2. Both IV therapy, as a ubiquitous and resource-intensive therapy, and the use of transfusion systems, dialysis systems, feeding systems, drainage systems, heart-lung machines, extracorporeal circulatory support systems, extracorporeal gas-exchange systems or ventilation systems should be integrated into modern electronic workflows. This means that the expected end of therapy, necessary syringe changes, feeding solution changes, drainage changes, etc., must be independently reported and requested by the fluid conduction and transfer system. 3. The fluid conduction and transfer system must be of sufficient robustness that a system test can allow, for example, the performance of infusion therapies and chemotherapies at home.

It is a further object of the invention to provide suitable components for such a fluid conduction and transfer system.

The object of the invention is primarily achieved by a connecting piece for fluidic coupling of medical appliances and/or medical lines.

A connecting piece in the context of the invention is a coupling device at the end of a fluid line or as an input or output of a medical appliance that can be mechanically coupled with an opposing counter-connecting piece or with an identical connecting piece in order to make the connecting pieces capable of fluid exchange. The invention relates to special connecting pieces from the field of medicine, which are specified in more detail below on the basis of the underlying standard. Connecting pieces according to the invention can be coupled with standard-conforming counter-connecting pieces to form a fluid connection.

Specifically, the invention relates to the following connecting pieces:
connectors of the ISO 80369 family of standards, in particular connector for intravascular or hypodermic applications according to ISO 80369-7, connector for enteral applications according to ISO 80369-3 and connectors for neuraxial applications according to ISO 80369-6,
plugs for an infusion bottle according to ISO 8536-2, crimp caps for an infusion bottle according to ISO 8536-3 and piercing spikes according to ISO 8536-4 for connection to an infusion bottle or a bag,
subcutaneous implanted ports according to ISO 10555-6 and non-coring cannula for coupling to such an implanted port according to ISO 10555-6,
conical connectors for anesthetic and respiratory equipment according to ISO 5356-1 and connectors according to ISO 5367,

connecting pieces for surgical wound drainage systems according to ISO 20697,
connecting pieces of an extracorporeal circuit of a hemodialysis system, hemodiafiltration system or hemofilter system as a blood port according to ISO 8637-1 (section 4.4.3, image 1 of the ISO standard) and connecting pieces for connection to blood ports for dialysis systems according to ISO 8637-2 (image 1 of the ISO standard), and
connecting pieces for internal connection of a dialysis system as a port for dialysis liquid according to ISO 8637-1 (section 4.4.4, image 2 of the ISO standard).

The standard specifications are based on the standards in force in July 2020.

A connecting piece according to the invention customarily allows the respective connecting pieces to create a liquid coupling between the connecting pieces. For this purpose, a connecting piece according to the invention has a fluid channel surrounded by a conduction section for circumferentially tight coupling in a plug-in direction to the counter-connecting piece for subsequent exchange of liquids and/or gases with the counter-connecting piece. Connecting pieces according to the invention are compatible with standard-conforming connecting pieces not according to the invention with regard to liquid conduction.

According to a particularly customary and preferred form of liquid coupling, conical conduction sections are provided on both sides on the connecting piece and on the counter-connecting piece, which are pushed together during coupling and hold by means of clamping.

In addition to the conduction section, a connecting piece according to the invention has a transmission device for data transmission and/or for transmission of electrical energy with the counter-connecting piece. Although separate transmission devices for data and for electrical energy are possible in principle, preference is given to a combined transmission device for data transmission and/or for transmission of electrical energy. Any transmission device elucidated in the context of this application is to be understood as meaning that it can also be a combined transmission device or a separate transmission device for electrical energy or data.

By means of its transmission device, a connecting piece according to the invention allows, in the context of the stated fluid conduction and transfer systems, establishment of connections between individual components, through which not only fluid exchange can take place, but also through which additionally electrical power and/or digital data can be exchanged between the connecting pieces.

The transmission of electrical energy or electrical data is needed in particular in order to supply energy to loads which follow the connecting piece in the fluid path. However, the function can also be a power line function from one connecting piece to a second connecting piece provided at the other end of a fluid line.

Data transmission at the connecting piece can serve the purpose of exchanging information, especially to check the topology, to exchange and possibly compare sensor data or to undertake other control functions or information acquisition functions. A very simple application is when data transmission is done in order to read production data of the connecting piece, such as, for example, the production batch or an expiration date. Another quite simple application is when a connecting piece is designed to send information about the permissible use of the connecting piece to a counter-connecting piece, so that misconnections can be detected.

The transmission device can be designed in different ways, as will be elucidated below. However, in each case, it is designed in such a way that a coupling process with respect to the fluid channels of the connecting piece and the counter-connecting piece also leads to the coupling of the data and/or energy transmission device at the same time.

Connecting pieces according to the invention in medical technology are usually predominantly designed as plastics parts which may adhere to limitations of the respective standards. The transmission device preferably comprises metallic or polymeric conductors which are especially preferably embedded in the plastic of the connecting piece. However, also possible are designs in which the conductors or contact surfaces connected thereto are attached on the outside of the connecting pieces.

Preferably, the connecting piece has a transmission device for data transmission and/or for transmission of electrical energy that is provided on the connecting piece in such a way that it can exchange data with or supply energy to a corresponding transmission device on the counter-connecting piece.

In this connection, it is preferred if the transmission device of the connecting piece has at least one coil for inductive coupling with the transmission device of the counter-connecting piece.

In inductive transmission, signals are transmitted between two coupled connecting pieces using electromagnetic induction. Relevant systems are known for energy transmission, especially through RFID tags and through the QI standard of the Wireless Power Consortium. Inductive data transmission and energy transmission is comparatively position-tolerant. This is especially advantageous in systems which cannot ensure a defined end position of the connecting pieces owing to the construction, for example a steep conical design of the coupling elements.

When the coils are arranged relative to one another in a defined manner, electromagnetic induction has a practical efficiency of over 50%. An efficiency of 80% can also be achieved. This means that, even with a serial arrangement of 5 pairs of connecting pieces, about 30% of the power is still retained.

The coils required for inductive coupling are preferably securely separated from the fluid, especially by being provided in an insulated cavity of the connecting piece. It is especially preferred if the coils are surrounded by a one-piece plastics part, which is preferably produced by an injection molding process with inserted coil.

Inductive transmission is not without any alternatives. The transmission device of the connecting piece can also have an electrode element for capacitive coupling with the transmission device of the counter-connecting piece and an electrode element there.

In principle, capacitive energy and data transmission is regarded as less tolerant of positional deviations than inductive energy and data transmission. However, in the case of connecting pieces according to the invention, since the relative position of two coupled connecting pieces is known very exactly depending on the type, capacitive coupling may also be a feasible option in the present application.

Furthermore, the transmission device of the connecting piece can also have at least one conductive contact surface or at least one conductive and preferably resilient contact element for galvanic coupling with the transmission device of the counter-connecting piece. Preferably, two contact surfaces or two contact elements are provided on the connecting piece.

This means that the connecting piece has provided thereon at least one contact surface which is exposed or is exposable by the coupling process, especially a metallic contact surface, which contact surface comes to rest on a correspondingly provided contact surface of the counter-connecting piece in the course of the process to couple connecting pieces. Preferably, one side is formed by a resilient contact element in order to allow position-tolerant coupling. In practice, galvanic coupling is regarded as usually disadvantageous, since the exposed contact surfaces can easily become soiled and the risk of unreliable data coupling is comparatively great. In addition, there is the problem of noninert surfaces which can be attacked by drugs and which can lead to biocompatibility problems. The advantages of galvanic coupling are, however, its simplicity and also the very high efficiency of energy transmission between the connecting pieces of almost 100%.

The transmission device is integrated in the connecting pieces in such a way that, when two connecting pieces are coupled, energy transmission and/or data transmission is possible without any interference. According to one possible construction, the transmission device has at least one planar coil, at least one planar electrode element and/or at least one planar conductive contact surface.

Such a planar element is understood to mean an element which, transversely to the plug-in direction, is at least three times as large as in the plug-in direction. Such a planar element can especially be arranged on or in a circumferential collar, which is preferably composed of plastic and/or integral with the stated support section. In the course of coupling, this circumferential collar is brought closer to another circumferential collar of the counter-connecting piece until the coil, the electrode element or the conductive contact surface is sufficiently close to allow data transmission between the elements facing one another.

The planar element is therefore preferably designed as an element surrounding the support section. In the case of a coil, this is preferably spiral-shaped, i.e., having a variable diameter. In the case of an electrode element for capacitive data or energy transmission and in the case of a contact surface for galvanic coupling, preference is given to a disk-type shape which has a central hole through which the fluid channel runs. In particular, in the case of a disk-shaped electrode element or a disk-shaped contact surface, two such elements can be provided in each case, which elements are concentric and have different diameters in order to allow two pole connections and/or bidirectional data transmission.

However, a design deviating from the planar design is preferred, namely one in which the transmission device has a helical coil surrounding the fluid channel, at least one annular or sleeve-shaped electrode element surrounding the fluid channel or at least one conductive contact surface surrounding the fluid channel as a ring or sleeve. An annular surface is understood here to mean a surface, the normal vector of which confines an angle of <30° with a radial direction. An inclination deviating from a pure annular surface or sleeve surface with a radial angle of 0° can arise in particular if the element is aligned according to the wall of a coupling cone.

With this type of design, a relevant or even primary extension of the elements in the plug-in direction is provided. In the case of a coil, it preferably has, at least sectionally, a cylindrical or conical shape which extends along the fluid channel. In the case of an annular or sleeve-shaped contact surface or an annular or sleeve-shaped electrode element, it has the above-defined shape, with preferably two corresponding elements being provided offset from one another in the plug-in direction.

A transmission device of this type is preferably attached by providing the connecting piece with a sleeve-shaped ring section, on the inner side of which, on the outer side of which or within which the helical coil, the annular electrode element or the annular conductive contact surface is provided.

Said sleeve-shaped ring section is preferably integral with the conduction section. It can thus be formed, for example, by a circumferential wall separated from the conduction section by a space. Said wall can, for example, additionally be a coupling section, on the inner side of which or on the outer side of which a coupling device is provided for mechanical coupling with the counter-connecting piece, especially an internal thread, an external thread, a bayonet slot or a bayonet cam for engaging a bayonet slot.

In particular, the ring section and the conduction section can, however, also be at least sectionally identical. The walls which directly delimit the fluid channel are, in this case, also carriers of the elements provided for transmission. In particular, depending on the type of connecting piece, the wall can be a sectionally conical wall which allows tight liquid coupling. Especially in the case of connecting pieces having such an at least sectionally conical wall, the end position of coupled connecting pieces is comparatively indeterminate, and so the transmission device in the sleeve-shaped ring section may be advantageous over a circumferential collar in the case of the planar design owing to the reproducible spacing and to sufficiently long coils in the plug-in direction. In the case of an at least sectionally conical wall in which a coil is provided, it can likewise accordingly be conical or else cylindrical. The conical design is preferred.

The use of the conduction section as a ring section which carries or surrounds the transmission device is sometimes advantageous over the use of a separate sleeve-shaped coupling section for accommodation of the transmission device, since it is thereby possible to use a rotatable coupling section, for example in the form of a union nut, which, as a rotatable element, would be connectable to a conductor of a fluid line only with additional effort.

The connecting piece is preferably made from plastic at least in part, and especially the stated conduction section, the circumferential collar or the sleeve-shaped ring section may be made from plastic. In this connection, what are preferred in each case are those plastics which can be found in the stated standards as recommendations or specifications.

In principle, what are possible are designs in which an electrical load or an integrated circuit is directly connected to the transmission device, without any further transmission of energy or data beyond the load or the integrated circuit. Usually, however, lines in the connecting piece are provided in the connecting piece and especially for the purpose of forwarding electrical energy or data. These are preferably metallic or polymeric conductors.

In the case of a connecting piece according to the invention, at least one output device can be provided, preferably in the form of an optical output device such as an LED. Such an output device can signal the status of the connecting piece directly on site. One example is the possibility of the output device revealing via lights or via the specific light color whether the connecting piece has been connected correctly. For example, in the case of a relatively long line composed of multiple parts, it would be possible to quickly detect whether all the connecting pieces have been connected correctly. If this is not the case, this can be recognized on the basis of the absent lighting of the corresponding LED or on the basis of a corresponding signal color.

Furthermore, in the case of a connecting piece according to the invention, at least one integrated circuit is preferably comprised. In particular, it can be integrated in a connecting piece. The integrated circuit is connected to the transmission device of a connecting piece in order to receive data or electrical energy.

In a particularly simple case, the integrated circuit can merely or primarily undertake communication tasks, especially communication with the data transmission devices of two connecting pieces. However, it can also undertake more complex functions and comprise especially digital or analog outputs or inputs. In this connection, the inputs can especially be connected to a sensor system integrated in the connecting piece or the fluid line, for example a sensor system for capturing the coupling state of the connecting piece or a sensor system for capturing a property of the fluid flowing through or its volumetric flow rate. The outputs can serve for control of actuators, the stated output device, especially in the form of an LED, also being an actuator in this sense.

The invention further relates to a set of at least two connecting pieces of the type described, which are designed according to one of the stated systems for mechanical coupling to one another, i.e., are usually male and female. In this connection, the two connecting pieces are designed in such a way that, through mechanical coupling, their respective fluid channels are brought into communicating connection with one another, and their respective transmission devices are brought into a position relative to one another that allows data transmission or transmission of electrical energy.

Such a set according to the invention usually does not have the two isolated connecting pieces, but rather two connecting pieces which are each part of a medical fluid line or a medical appliance.

The invention further relates to a medical fluid line for transferring liquids. Said fluid line has a conduction body having an elongated shape, through which a fluid channel for the fluid or fluid mixture passes. It can especially be a rigid pipe body or a flexible tube body. A connecting piece of the type described above is provided at least at a proximal end of the conduction body, but preferably both at a proximal and an opposite distal end of the conduction body, so that the channel of the conduction body communicates with the fluid channel of the connecting piece(s). Furthermore, designs of a fluid line having more than two ends, for example a fluid line having a Y configuration, are also conceivable. In such a case, corresponding connecting pieces are preferably provided at all the ends of the fluid line.

If the fluid line is provided with at least two connecting pieces, they can be identical or be designed to be diametrically opposed in the form of a male and a female connecting piece.

An electrically conductive conductor for conducting digital data and/or for conducting electrical energy is preferably also provided along the conduction body with the fluid channel provided therein. Various designs are conceivable here. For instance, in the simplest case, the conduction body and the conductor can merely run parallel to one another, but without being connected to one another. It is also possible that the conductor is formed separately from the conduction body, but is connected to the conduction body along the extension of the conduction body by coupling elements or by helical wrapping around the conduction body. However, what are considered to be particularly preferred are integrated solutions in which the conduction body and the line, i.e., the electrically conductive conductor, are firmly connected to one another, so that they cannot be separated from one another, at least not without tools. In particular, the conduction body and the line can be inserted into a common sheath for this purpose. Alternatively, the electrical line can be inserted into a sheath which directly forms the channel of the conduction body.

Since a conduction body designed as a tube body has quite a high degree of stretchability, the conductor is preferably designed and/or shaped in such a way that it is not damaged by stretching or deformation of the conduction body. This can, for example, be achieved by a polymeric conductor. It is also possible, through the arrangement of the conductor, to create a kind of reserve which allows elongation of the tube body using the reserve of the conductor. Preferably, the conductor in the region of the tube body is therefore at least 10% longer than the tube body itself, especially preferably even 50% longer. The conductor can surround the fluid channel of the tube body, for example in a helical manner. A longer conductor is also advantageous if the tube body, as part of a peristaltic pump, is repeatedly compressed and relaxed as intended. In such a case, it is, however, especially advantageous to sectionally configure the tube body without a conductor, as will be elucidated below.

According to the usual way of production, a connecting piece according to the invention is used as a discrete connecting piece that is connected to, for example molded on, the conduction body. However, also possible is a design in which the proximal end or the distal end of the conduction body integrally forms the conduction section of at least one connecting piece. Here, accordingly, the end of the conduction body directly forms the conduction section and, at the same time, the transmission device is preferably likewise directly provided on the conduction body, especially integrated in the wall thereof.

Usually, the coupling-in of energy and/or data transmission takes place in the region of a connecting piece of the fluid line. However, it is also possible to provide a fluid line according to the invention with a connecting device intended for this purpose in a central region and hence especially at a distance from terminally provided connecting pieces. This is of particular benefit in connection with the use of a pumping device, which will be described below, as a master of a data system. Such pumping devices are commonly designed as peristaltic pumps and are intended for insertion of conduction bodies, which are externally mechanically deformed for the purpose of conveying liquid. If at least one connecting device is provided in the central region described, the former can be externally supplied with data and/or electrical energy after insertion of the conduction body.

For this purpose, it is considered to be particularly advantageous if the at least one connecting device for coupling-in/coupling-out of electrical energy or data is provided with a carrier which extends radially from the conduction body and in which or on which the actual connecting device, i.e., a coil for example, is provided. Such a coil is furthermore preferably arranged eccentrically to the fluid channel, so that it does not thus surround the fluid channel, but is arranged laterally offset thereto. It can thus be used particularly advantageously for energy transmission or data transmission with a counter-coil which is provided for this purpose in the pumping device.

While an inductive transmission device is preferably provided in connecting pieces according to the invention in order to avoid contact between current-carrying parts and the fluid, providing galvanic coupling is considered to be a plausible option in the case of the connecting device described in the central region. What are then provided on the outer side of the tube body, especially on the radially extending carrier described, are conductive coupling surfaces which, after insertion of the fluid line into the pumping device, are in contact with corresponding contact surfaces there.

If the tube body of the fluid line is compressed as intended by a pumping device during pumping, this can quickly lead to damage to the conductor along the fluid line. In order to avoid this, a fluid line according to the invention can be without a conductor in a subsection in a central region, i.e., between ends of the fluid line and connecting pieces there that are opposite one another. Said subsection can then be repeatedly compressed for the purpose of pumping without damaging the conductor.

If connecting pieces according to the invention having a transmission device are provided at the two ends of the fluid line, the fluid line preferably has two connecting devices of the type described in the central region, specifically on the two sides of the subsection provided for pumping.

As with a connecting piece according to the invention, at least one output device can also be provided in a fluid line according to the invention. Preferably, such an output device is provided on a connecting piece or on two connecting pieces at opposite ends of the fluid line.

Furthermore, in a fluid line according to the invention, at least one integrated circuit can also be comprised, which integrated circuit can especially preferably be integrated in a connecting piece of the fluid line. However, in the case of fluid lines having two or more connecting pieces, preferably only one of the connecting pieces is provided with an integrated circuit. Via conductors along the conduction body, the other connecting pieces are then likewise connected to said integrated circuit. However, also possible are designs in which different connecting pieces of a fluid line or else of a medical appliance can have separate integrated circuits which do not need to be connected to one another for data transmission. For example, two different connecting pieces of a fluid line or else of a medical appliance could each be designed for transmission of an identifier by means of separate integrated circuits, said identifiers allowing a connected counter-connecting piece to detect whether the coupling of the connecting pieces is permissible or not.

The integrated circuit is connected to the transmission device of a connecting piece in order to receive data or electrical energy. In a particularly simple case, it can merely or primarily undertake communication tasks, especially communication with the data transmission devices of two connecting pieces or with components connected thereto. However, it can also undertake more complex functions and comprise especially digital or analog outputs or inputs. In this connection, the inputs can especially be connected to a sensor system integrated in the connecting piece or the fluid line, for example a sensor system for capturing the coupling state of the connecting piece or a sensor system for capturing a property of the fluid flowing through or its volumetric flow rate. The outputs can serve for control of actuators, the stated output device, especially in the form of an LED, also being an actuator in this sense.

The invention further relates to a fluid-guiding medical appliance which is designed for receiving, conducting and/or delivering fluids and, furthermore, for receiving, conducting and/or delivering data and/or electrical energy. According to the invention, said appliance has a connecting piece of the described design for combined receiving, conduction and/or delivery of data and/or electrical energy and fluid. In the simplest case, such an appliance is formed by a simple fluid line of the type described.

In particular, this however also encompasses more complex appliances which serve for temporarily storing liquid or for keeping it available or which have at least one actuator for controlling the fluid and/or applying pressure thereto. Such an actuator can be, for example, an electrically controllable valve or a pump. The actuator can be supplied with energy via the electrical energy supplied by the connecting piece or via a separate power supply. Control data for controlling the appliance can be supplied by the connecting piece or be sent to connected appliances via the connecting piece.

An appliance of the type described can furthermore have at least one sensor, the sensor data of which can preferably be sent to further appliances via the connecting piece. The supply of energy can also be effected here via the electrical energy supplied by the connecting piece. The sensor can be provided for capturing various variables. What is particularly advantageous is a design in which the sensor is designed for capturing a property of the fluid, for example the temperature, or for capturing the volumetric flow rate of the fluid. The sensor can, however, also be provided for capturing ambient parameters on the body or in the body of a patient, for example as a temperature sensor, pressure sensor or sensor for capturing oxygen saturation. Such a sensor can be, for example, part of a catheter introduced into the patient or part of an implanted port.

Preferably, the appliance has at least one integrated circuit which is connected to the connecting piece for the purpose of supplying electrical energy or digital data. Said integrated circuit can also be operated with energy supplied by the connecting piece and/or exchange data with other appliances via the connecting piece. The integrated circuit serves especially for structuring electronic communication with neighboring fluid-conducting components according to the invention. The integrated circuit can locally process in the appliance the data of the described sensor and/or locally control the described actuator. In particular, the integrated circuit can be used to implement a closed control loop in the appliance using the sensor and the actuator. Furthermore, the integrated circuit serves especially for communication with external components connected via the connecting piece according to the invention.

In most cases, it is appropriate, in the case of an appliance having at least two connecting pieces according to the invention, if the at least two transmission devices thereof are connected to a common integrated circuit. It is preferably capable of differentiating the at least two connecting pieces and/or sending specific, different connection identifiers for the two connecting pieces by means of the transmission device. Alternatively, it is however also possible to provide each connecting piece with its own integrated circuit, which is designed for sending only one connection identifier, which, however, is likewise specific and unique for the connecting piece.

A medical appliance according to the invention can be realized in the form of very different appliances.

The appliance can, for example, be designed as a conveying unit, and this is to be understood to mean that the appliance is designed for application of pressure to the fluid. Such a conveying unit can especially be designed as a pumping device and, as such, be configured with a plunger. In particular, the pumping device can, however, be a peristaltic pumping device in which an external force is periodically applied to a fluid tube for onward transfer of the liquid in the tube. A conveying unit according to the invention with a peristaltic pump is preferably designed for insertion of a fluid line according to the invention of the type described.

As will also be elucidated below, a medical appliance according to the invention in the form of a conveying device and especially a circuit integrated therein is also suitable for being a master in data communication with further components connected to the conveying device. Such a master controls communication via the transmission devices and sends, for example, control commands or requests for data transmission to the other components.

An appliance according to the invention can furthermore be designed as a central venous catheter or peripheral venous catheter. Such a catheter forms a thin line to a vein of the patient and may be provided for temporary residence on the patient. In many cases, such catheters have a plurality of connecting pieces which allow the simultaneous supply of different fluids and fluid mixtures. A catheter having connecting pieces according to the invention allows storage in its integrated circuit of the fluid sources which have been connected thereto in order to detect, on the basis thereof, potential incompatibilities.

The appliance can furthermore be designed as a multi-way valve or as a valve bank. Such a multi-way valve or such a valve bank has at least three connecting pieces, which are preferably all designed as connecting pieces according to the invention, and controls the fluid flow in the appliance to the connecting pieces into the position of a rotary and directional valve. A multi-way valve or valve bank according to the invention preferably senses the position of at least one directional regulating valve and, by means of its integrated circuit and the connecting pieces according to the invention, can identify the connected appliances and capture its valve position and connection and pass on the information via the connecting pieces according to the invention, especially to one or more masters which are preferably formed by the conveying devices described.

The appliance can furthermore be designed as a hemodialysis cartridge, a hemodiafilter cartridge, a hemofilter or a hemoconcentrator cartridge. In the context of this disclosure, these units are referred to under the term dialysis cartridge.

The appliance can furthermore be designed as an auxiliary port device and has as such a fluid line provided with a connecting piece on both sides, between which there is provided an auxiliary port for ad hoc addition of a fluid. Such an auxiliary port offers the possibility of a temporary connection, for example for injection of drugs via needle- or valve-based access ports on the fluid system. The appliance can monitor this injection port owing to the continuous supply of energy to the appliance. If the connecting piece of the auxiliary port is designed as a connecting piece according to the invention, the appliance can additionally communicate with these injection appliances, for example with a syringe having a connecting piece according to the invention, and thus, for example, retrieve the type of drug and also information about the dose and the manufacturer.

The appliance can furthermore be designed as a filter appliance.

The appliance can furthermore be designed as a ventilation tube.

The object of the invention is also achieved by an intelligent medical fluid conduction and transfer system which has an access unit for administrating a fluid or a fluid mixture into a patient or for withdrawing a fluid from a patient and which has at least one liquid container for providing or accommodating the fluid. In this connection, use is also made of the described connecting pieces, which are used on the access unit and/or on the liquid container and/or on fluid lines between liquid container and access unit.

In addition to the container and the access unit, the system preferably comprises at least one further appliance of the type described above, which can be a conveying device for example.

A system in this context thus comprises at least the components absolutely necessary for supply of fluid or withdrawal of fluid, the basis of data and/or energy transmission being created through the use of connecting pieces according to the invention, especially also through the use of fluid lines and/or appliances according to the invention.

The system especially comprises a medical appliance, for example a fluid pump having an integrated circuit which can retrieve data of the liquid container or the access unit via the data connection created by the connecting piece.

In the system, the medical appliance can form a master which is designed for communication with other components, or the integrated circuits of other components, of the system, for example the liquid container or the access unit. However, the master can also be separate from an appliance according to the invention, i.e., cannot itself have components which directly guide fluid.

A system can have multiple masters, for example multiple infusion pumps which, with regard to the components upstream and/or downstream thereof in the liquid path, each control communication with the respective integrated circuits of the components. If a system has multiple masters, they are preferably connected to one another via a separate network, for example via Ethernet or a fieldbus, in order to exchange data of their respective branches of the system or to coordinate control tasks in the different branches of the system with one another.

In principle, multiple masters in a system can also undertake different tasks to the effect that one master supplies the system with electrical energy, whereas another master controls data communication. However, it is preferred if a common master unit serves both for supply of energy to the other components and for control of communication with said other components.

Depending on the type of system, different components or the integrated circuits thereof are suitable as master. In the case of an infusion system, the master is preferably formed by an infusion pump of the system. In the case of a dialysis system, the master is preferably formed by a dialysis machine of the system. In the case of a feeding system, the master is preferably formed by a feeding pump of the system. In the case of a drainage system, the master is preferably formed by a drainage pump of the system. In the case of a ventilation system, the master is preferably formed by a ventilation machine of the system.

Furthermore, the system and especially the master of the system are preferably designed for communication with a server via a data network, especially via an intranet or Ethernet. This can especially be done in order to retrieve data relating to an identification code of an integrated circuit. For instance, supplementary information can be assigned to such an identification code stored in the integrated circuits of the system, for example a use-by date, a batch number or specification data of the fluid line or of the appliance in which the integrated circuit is provided.

Also appropriate is communication with a server in order thereby to retrieve or supplement the medical file using a patient or hospital information system. The data retrieval can especially serve the purpose of comparing the patient care provided by the system with information in the medical file. Supplementing the medical file means that the care provided by the system can be documented automatically.

The object of the invention is also achieved by an intelligent medical fluid conduction and transfer system. Said system comprises at least one fluid source for providing a medically relevant fluid or fluid mixture and also an access unit for administering the medically relevant fluid or fluid mixture into a patient. The system comprises furthermore a connecting conductor system which connects the fluid source and the access unit and is designed for conduction and transfer of the medically relevant fluid or fluid mixture from the fluid source to the patient, the connecting conductor system preferably having at least one regulating transfer element.

The intelligent medical fluid conduction and transfer system is preferably equipped with at least one of the components as described above.

The components of the medical fluid conduction and transfer system are designed at least in part to determine and exchange information about their status and to report said information to a master. To transmit said information, the connecting conductor system is designed to transmit and forward said information and/or to provide an energy supply for the components of the medical fluid conduction and transfer system, with the result that the components of the medical fluid conduction and transfer system form their own information infrastructure and wherein a network topology of the connecting conductor system and of a fluid flow in the medical fluid conduction and transfer system can be determined by means of said information infrastructure.

To convey the medically relevant fluid mixture from the fluid source to the access unit on the patient, a conveying unit for conveying the medically relevant fluid mixture is preferably formed between the fluid source and the access unit. Such a conveying unit can be, for example, an infusion pump in an infusion system. In a ventilator, the pressure is built up via a pump or through the acting media pressure of the ventilation gases.

In drainage situations for wound secretion and urine, the natural production of fluid by the body is used to drive the fluid flow. In drainage situations, the fluid flow is reversed, i.e., the fluid source is in the body of the patient and transfers the body secretion into a reservoir outside the body. The fluid flow can also be supported by a pump as in the case of thoracic drainage. The advantages of the information infrastructure according to the invention in the fluid conduction systems and distribution elements likewise become important here, since the integrity and topology of the drainage system can be checked by means of said infrastructure. The drainage pump, for example, can act as a master.

In dialysis systems, heart-lung machines or extracorporeal gas-exchange systems, the medically relevant fluids or fluid mixtures are driven by pumps or by natural media pressure (blood pressure).

In order to ensure the transmission of information and energy, metallic or polymeric conductor paths are integrated at least in some of the and preferably in all of the fluid-conducting components of the fluid conduction and transfer system, i.e., including in the connecting conductor system. As a result, via the connecting lines, it is possible not only to effect the medically relevant fluid mixtures from a fluid source to an access unit into or out of the patient, but also, in parallel, to transmit or transfer data, information and/or energy between the components of the fluid conduction and transfer system.

In the context of this invention, a fluid or fluid mixture covers substances which are either gaseous or liquid, similarly suspensions and aerosols. A connecting conductor system in the context of the invention is understood to mean infusion tubes, IV catheters, ventilation tubes, connecting tubes and connectors.

Via the information infrastructure, it is possible to advance and transfer actuator information and commands for changing the flow topology or information-display content. The information infrastructure in the fluid conduction and transfer system according to the invention can also be located within medical appliances—for example, infusion pumps, dialysis machines, heart-lung machines—or within the patient, i.e., in the access unit.

Components of the fluid conduction and transfer system that are designed to determine their status and report it to a master are understood as elements which are active in terms of information technology. This means that said elements have electronics through which identification (type, serial number, unique identifier, lumen identification in the case of a multilumen catheter), state (position of three-way valves, catheter clamp open, fill level of infusion containers), properties (drug in the lumen, manufacturing details) and sensor data from the patient are transmitted. Said components can have their own power source. For example, the infusion pump in an infusion system can be operated via a grid connection or by means of a battery. The other components can likewise have a power source.

Because the components can determine and report their status, the elements of an IV therapy, a dialysis system, a feeding system, a drainage system, a heart-lung machine, an extracorporeal circulatory system, an extracorporeal gas-exchange system or a ventilation system are identifiable. This means that, for example, the network topology on the patient side is determinable for each drug. In addition, the connections, multi-way valves, valve banks, etc., can be uniquely identified by the type thereof, a serial number or an ID uniquely assigned thereto. The advantage is that pharmaceutical drug intolerances, for example, can be identified through the unique identification of the components and hence through the determination of the network topology. If the fluid conduction and transfer system according to the invention in the form of an infusion system is connected to a drug and compatibility database, it is thus possible to identify and prevent drug instabilities and precipitations in the infusion system. The same applies to transfusion systems, dialysis systems, feeding systems, drainage systems, heart-lung machines, extracorporeal circulatory systems, extracorporeal gas-exchange systems and ventilation systems. The network topology identified by the respective system can also be compared with a prescribed therapy topology and raise an alarm in the event of incorrect drug administration or the like.

A further advantage of the fluid conduction and transfer system according to the invention is that the effective physical connections of the fluid conduction and transfer system can be checked and thus the probability of leakage can be reduced. Similarly, flow rate can be measured in the immediate vicinity of the patient and compared with the parameters of a conveying unit, for example an infusion pump, and checked for plausibility.

The components of the fluid conduction and transfer system each have a coupling device for coupling-in and coupling-out of information and/or for coupling-in of energy. In the context of the present invention, a coupling device is understood to mean a connection point which makes it possible to transmit or transfer data from one component of the fluid conduction and transfer system, via the connecting conductor system, to a further component of the fluid conduction and transfer system or to a master.

Such a coupling device or connection is, for example, designed in such a way that compatibility with the known Luer system is maintained. Coupling can, for example, be effected via a Luer lock on a Luer lock or via a Luer slip on a Luer slip or via a Luer slip on a Luer lock or via a piercing connection from an infusion container in the form of a bottle or a bag to the connecting conductor or via a piercing connection from a port needle to an implemented port, the respective coupling being expanded by electronics which allow transmission of data and energy. The regulating transfer elements are also coupling devices in the context of the invention. The same principle applies to the connections in feeding systems, ventilation systems, circulatory support systems, oxygenation systems, dialysis systems and drainage systems, the connection geometries typical of the respective applications being used and being expanded by a coupling functionality for information and energy. For this purpose, the couplings and/or transfer elements known from the prior art are likewise expanded with electronics which allow transmission of data and energy. An expanded transfer element having corresponding electronics for coupling-in of energy and information is comparable to a hub which distributes and transfers energy or information signals to multiple components of the fluid conduction and transfer system. A transfer element can be formed at various points in the fluid conduction and transfer system, depending on where there is a need to regulate the medically relevant fluid mixture to be administered.

The coupling devices are also understood as nodes in the network topology of the fluid conduction and transfer system. The signal is processed and/or switched at each node in the network. This allows enumeration of the topology—similar to a USB tree for example—and routing of data packets to addressable nodes. By means of schematically planned, staggered interrogation of individual components (time-slot method) or by means of frequency-division multiplexing methods or the like, there is thus also, firstly, reduction of the energy demand and, secondly, prevention of the collision of data packets.

The transmission and forwarding of information and/or supply of energy between the components of the fluid conduction and transfer system and the connecting conductor system via the coupling device can be effected via an electrical conductor in a frequency range greater than 100 kHz. The transmission of energy and data packets is effected in a frequency range which does not collide with the action potential and the latency times of cells or adversely affect them. Frequency ranges used for electrosurgery are omitted in order to avoid signal interference.

In a further embodiment of the fluid conduction and transfer system according to the invention, it is very advantageous if the connection is effected in a contactless manner by inductive or capacitive coupling. The connection points of the components communicating with one another are galvanically separated from one another for this purpose. The physical connection of the components is still effected via the Luer system or via the medically appropriate connectors specific for the particular application. However, the parts to be connected are designed in such a way that transmission is effected by inductive or capacitive coupling, i.e., by the mutual electromagnetic influencing of two or more spatially adjacent electrical circuits.

The transmission and forwarding of information and/or supply of energy between the components of the fluid conduction and transfer system and the respective connecting lines of the connecting conductor system can also be effected from a combination of the types of transmission, via an electrical conductor, inductively and capacitively.

For all the types of transmission, lines of the connecting conductor system lying next to one another must not exhibit relevant interference with one another on the basis of their design, even in complex therapy situations. Modulated carrier frequencies, similar to common NFC technologies, are therefore used for data and energy transmission. They are appropriately chosen with regard to amplitude and frequency.

In contrast to the elements which are active in terms of information technology, components of the fluid conduction and transfer system which merely transmit information and energy via inductive or capacitive coupling are understood as elements which are passive in terms information technology.

In one embodiment of the fluid conduction and transfer system according to the invention, the master is formed by the conveying unit, the coupling unit or in a cloud.

The fluid conduction and transfer system according to the invention can have multiple masters. It is advantageous if each conveying unit, for example an infusion pump, and/or the connecting conductor system and/or transfer elements is designed to receive data of the system, to evaluate said data and to respond accordingly to said data. In infusion systems, it is, for example, known and common practice that exactly one infusion pump is used for each drug or for each infusion solution. In addition, grid operation or battery operation of the infusion pump means that it offers the advantage of always having sufficient energy reserves available for communication with the remaining components of the infusion system. The system-related directionality of the infusion pump also specifies a clear communication logic, comprising the following steps: i) query in the proximal direction about which drug or which infusion solution is assigned to the pump; using sensor information ascertained via a corresponding sensor system in the drug containers, it is possible to determine the status of the infusion solution such as fill level, manufacturing details, batches, expiration date and unique ID. ii) query in the distal direction about which transfer elements are present; which IV line, which lumen and which patient is connected to the infusion system. Sensor data such as body temperature and oxygen saturation can be acquired and evaluated. The same applies to the embodiment of the fluid conduction and transfer system as a transfusion system, dialysis system, feeding system, drainage system, heart-lung machine, extracorporeal circulatory system, extracorporeal gas-exchange system or ventilation system.

Furthermore, it is advantageous if the master is designed to carry out a unidirectional or bidirectional exchange of information with a patient or hospital information system. What is possible as a result is that the fluid conduction and transfer system can independently indicate and request, for example, necessary syringe changes or an expected end of therapy and thereby eases the burden of medical personnel.

In addition to direct data utilization for control and monitoring of infusion therapy, transfusion therapy, dialysis therapy, feeding therapy, drainage therapy or ventilation therapy, they can thus also undergo automatic, high-quality documentation. This allows full traceability.

By means of the fluid conduction and transfer system according to the invention, integrity from the fluid source up to the patient can be independently validated by the system. Misconnections and drug incompatibilities are detected, and therapy can be completely and reliably documented without great personnel effort. Using the fluid conduction and transfer system according to the invention, it can be ensured that hospital resources are saved and the quality of patient management is increased, for example by the fluid conduction and transfer system being able to independently report and request the expected end of therapy and necessary syringe changes.

A further advantage of the fluid conduction and transfer system according to the invention is also its modular structure. The system is easily expandable by further conveying units, fluid sources and connecting conductors in the connecting conductor system, which can be added to the network topology and information infrastructure without any difficulties.

According to the invention, there is likewise proposed a computer program product which comprises commands which, upon execution of the program by a computer, prompt said computer to analyze the network topology of the fluid conduction and transfer system, to check the integrity of the fluid conduction and transfer system and to use them for documentation in a patient file. Said computer program product can be operated via a tablet carried by the physician or medical personnel. The concept of operating the system can also be simplified in such a way that said system is suitable for carrying out, for example, infusion therapies and chemotherapies at home.

In an advantageous application, the medical fluid conduction and transfer system according to the invention is designed as a measurement system having a fluid column between the inside of the body of a patient and an extracorporeal measurement system for arterial or venous blood pressure. In this connection, a column of liquid between an intra-arterial catheter in the patient and an extracorporeal pressure measurement system for the transmission of the blood pressure is used for the intra-arterial pressure measurement. The information infrastructure according to the invention in the tubes, connectors and three-way valves required therefor also allows here an automatic integrity check, the detection of misconnections and the direct reporting of flushing processes in the system and the measurement unit. This avoids measurement errors during the flush cycles or blood sampling from the system, since the master can detect these events from the dynamic topology (such as, for example, the position of three-way valves) and communicate said events to the measurement system. This principle applies analogously to invasive venous pressure measurement.

In summary, the fluid conduction and transfer system according to the invention represents an information infrastructure in infusion therapy, transfusion therapy, dialysis therapy, feeding therapy, drainage therapy and ventilation therapy that makes it possible to independently validate integrity from the fluid source up to the patient, to detect misconnections and drug incompatibilities and to provide validated documentation of the therapy. Therefore, it is also referred to as an intelligent fluid conduction and transfer system. The system according to the invention offers full connectivity, there being largely no need for physical contact surfaces. This means that the system is also ideally sterilizable, and no new biocompatibility problems arise, since contact with the drugs continues to occur via the glass and polymer materials tried and tested for this purpose. Furthermore, the system is fully backwards-compatible through integration in the Luer standard and offers itself as a medium-term industry standard for “smart fluid conduction and transfer system technology”.

### BRIEF DESCRIPTION OF THE DRAWINGS

Further advantages and aspects of the invention are revealed by the claims and by the following description of preferred exemplary embodiments of the invention, which are elucidated below with reference to the figures.

FIG. 1 shows a schematic depiction of a network topology of a fluid conduction and transfer system according to the invention using the example of an infusion system.

FIG. 2 shows, in the form of a schematic depiction, a network topology of the fluid conduction and transfer system according to the invention using the example of a dialysis system.

FIG. 3 shows, in the form of a schematic depiction, a network topology of the fluid conduction and transfer system according to the invention using the example of a wound drainage system.

FIG. 4 shows, in the form of a schematic depiction, a network topology of the fluid conduction and transfer system according to the invention using the example of a ventilation system.

FIG. 5 shows two connecting pieces in the form of a Luer lock having a planar inductive transmission device.

FIGS. 6A to 6C show connecting pieces in the form of Luer couplings having different planar transmission devices.

FIGS. 7A to 7C show connecting pieces in the form of Luer couplings having sleeve-shaped transmission devices in the region of a conduction section.

FIGS. 8A to 8C show connecting pieces in the form of Luer couplings having sleeve-shaped transmission devices in the region of a coupling device for mechanical coupling.

FIG. 9A shows the connection between, firstly, a crimp cap of an infusion bag or a bottle and, secondly, an infusion tube having expanded electronics by means of inductive coupling.

FIG. 9B shows the connection between, firstly, a closure plug of an infusion bag or a bottle and, secondly, an infusion tube having expanded electronics by means of inductive coupling.

FIG. 10 shows the connection between an implanted port and a port needle having expanded electronics.

FIG. 11 shows a connecting piece formed by one end of a tube, in particular as part of a drainage system.

FIG. 12 shows connecting pieces of a ventilation system.

FIG. 13 shows connectors of a ventilation system.

FIG. 14 shows connecting pieces on blood ports of a dialysis cartridge.

FIG. 15 shows connecting pieces on dialysate ports of a dialysis cartridge.

FIGS. 16A and 16B show different types of design of a conduction body having a conductor routed in parallel.

FIG. 17 shows an infusion pump as a master with coupling-in of energy and coupling-in and coupling-out of information.

FIG. 18 shows a central venous catheter.

FIG. 19 shows a peripheral catheter.

FIG. 20 shows a three-way valve.

FIG. 21 shows a valve bank.

FIG. 22 shows a dialysis cartridge.

FIG. 23 shows an auxiliary port device.

FIG. 24 shows a filter unit.

FIG. 25 shows a tube unit for a peristaltic pump.

### DETAILED DESCRIPTION OF THE EXEMPLARY EMBODIMENTS

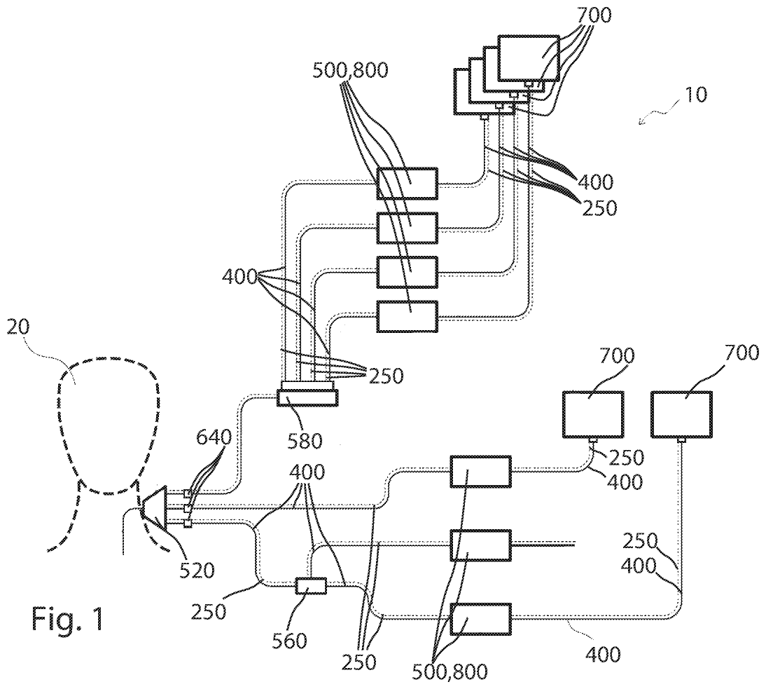

FIG. 1 shows the schematic depiction of a network topology of the fluid conduction and transfer system 10 according to the invention in the form of an infusion system. In contrast to known infusion system topologies, the fluid lines 400 between the components 700, 500, 580, 560, 520 of the system 10 and the fluid-guiding components 700, 500, 580, 560, 520 themselves have metallic or polymeric conductor paths 250 that make it possible to forward and exchange data/information and/or energy. The components of the infusion system 10 are each provided with electronics, so that the individual components—such as the transfer elements 560, 580 (e.g., three-way valves 560 or a valve bank 580) or fluid sources 700 (such as infusion bags) or conveying units 500 (such as infusion pumps)—are capable of identifying themselves and of determining and reporting their status.

By way of example, the intelligent infusion system 10 allows the functionality of detecting faulty connections in the infusion system. By means of computer code integrated according to the invention, the overall system can determine its topology and hence establish a deviation of the prescribed drugs and the medically prescribed therapy scheme from the actual topology of the infusion system. The connection of a drug from a fluid source 700 to the correct patient 20 is included in this check. Since the components of the infusion system on the patient's side—including central venous catheter 520—have been implanted in the patient 20, they allow a unique identification of the patient 20 and hence allow checking of whether a drug or an infusion solution from a fluid source 700—i.e., an infusion bag or an infusion bottle—will be administered to the correct patient 20.

By way of example, the intelligent infusion system 10 allows the functionality of detecting incompatibilities between drugs or between drugs and infusion solutions at the point in the infusion system 10 where the fluid flows meet. To this end, the computer program accesses compatibility databases and compares the infusion topology found with said databases.

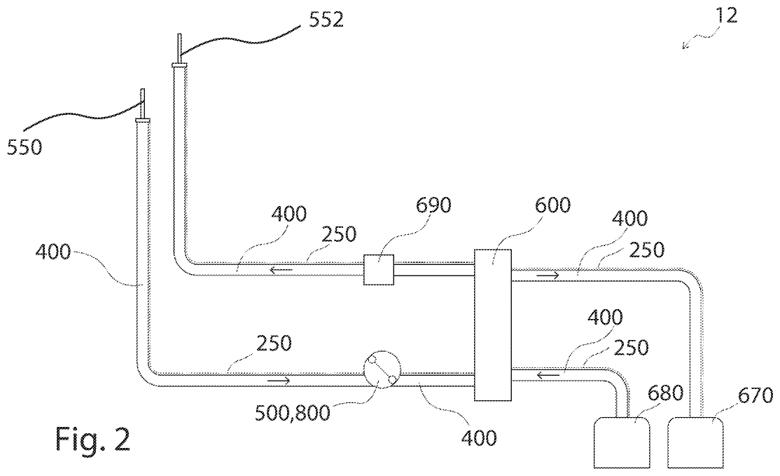

FIG. 2 shows the fluid conduction and transfer system according to the invention in the form of a dialysis system 12. An equitopological information infrastructure is integrated in all the components of the blood inflow—i.e., from the venous line 550 into the dialysis system 12 comprising the blood pump 500, the blood line 400 to the blood pump 500 and dialysis cartridge 600, the respective connectors (not depicted) and the dialysis cartridge 600 and also the return flow composed of blood line 400 to the patient, the air trap 690 and the venous line 552—to the patient. An information infrastructure is also integrated in all the components of the dialysis system 12, such as the dialysis solution 680 with the supplying dialysis line 400 to the dialysis cartridge 600, and the dialysate container 670 with the dialysate line 400. The connections between the components are, for example, realized as in FIG. 2. As a result, the integrity of said connections to the patient and within the dialysis machine can be verified and misconnections can be detected. The blood pump 500, for example, can serve as a master 800 and as an energy feed and information interface.

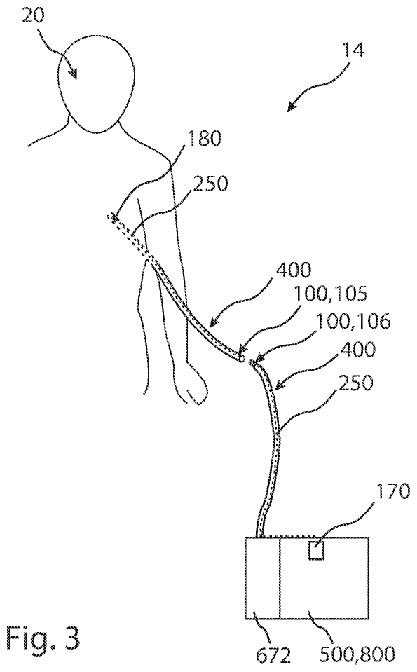

FIG. 3 shows the fluid conduction and transfer system according to the invention in the form of a wound drainage system 14. It has a fluid line 400 which is guided into the body of the patient 20. Said fluid line, which is depicted at the top of the figure, is connected to a second fluid line 400, which is connected to a unit composed of secretion container 672 and suction pump 500. The suction pump 500 generates a negative pressure in the secretion container 672 and, as a result, liquid is sucked out of the body of the patient through the fluid lines 400.

The two fluid lines 400 are designed as fluid lines according to the invention and therefore have connecting pieces 105 and 106, which connect not only the fluid channel thereof, but also the conductors 250 provided on both fluid lines 400. On the patient's side, the conductor 250 leads to the distal end of the fluid line 400 ending in the body of the patient 20. A sensor 180 is provided here, which can acquire body data of the patient 20. An integrated circuit 170 of the suction pump 500 is connected to the sensor 180 via the conductors 250 and via the connecting pieces 100 and can thus retrieve the body data and optionally store said data in a patient file via a network connection that is not depicted.

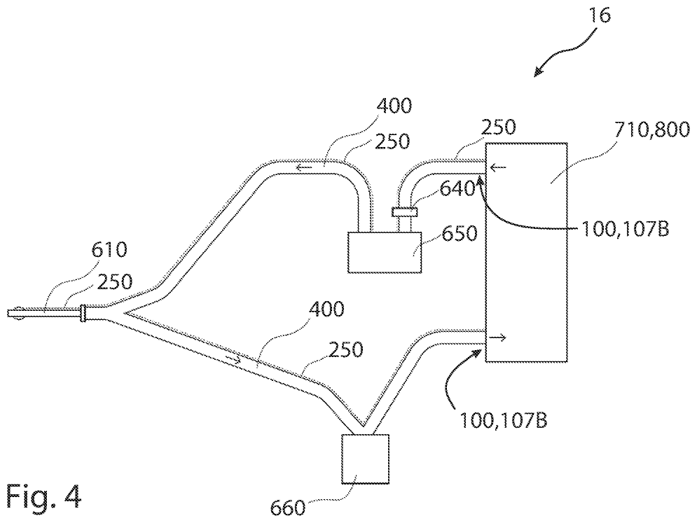

FIG. 4 shows the fluid conduction and transfer system according to the invention in the form of a ventilation system 16. Owing to the conductors 250 in the ventilation tubes—such as the inhalation limb 400 and the exhalation limb 400—there is an additional possibility of checking for correct connections, i.e., the correct topology when fitting humidifiers 650, filters 640 and liquid traps 660. The connections of the information infrastructure can, for example, be realized inductively according to FIGS. 12 and 13 with adaptation to the geometry of the connection standards in the ventilation system. Sensor data from the ventilation machine 710 or the tube 610 can be transmitted via the information infrastructure 250 without additional cables. The ventilation machine 710, for example, can serve as a master 800 and as an energy feed and information interface.

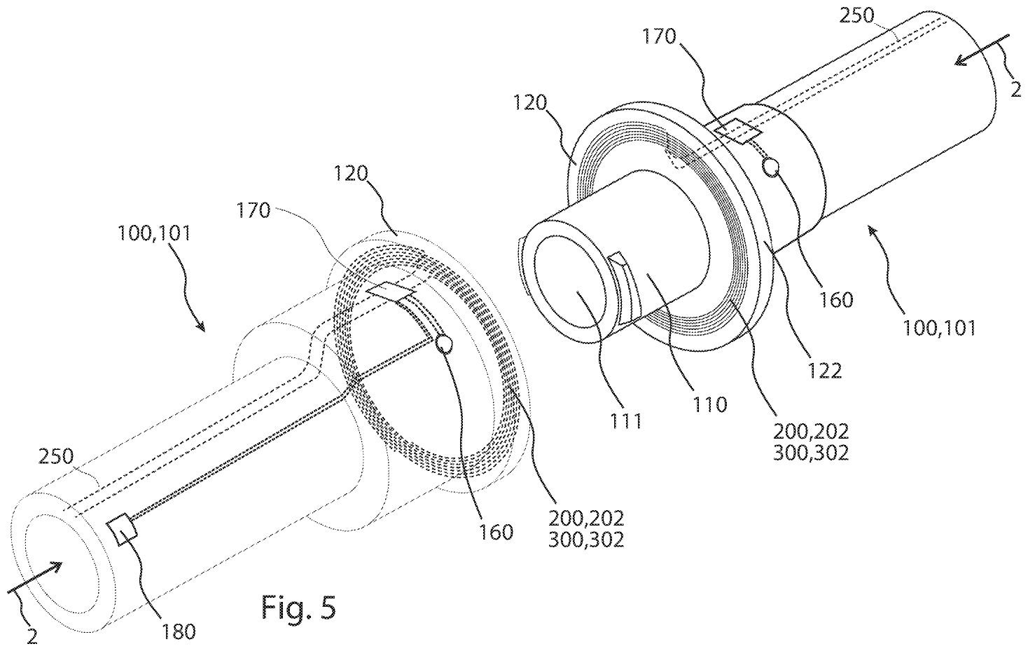

FIG. 5 shows a simple Luer lock according to ISO 80369-7, which realizes a standardized mechanical connection between a first and a second connecting piece 100, 101 of the connecting conductor system. The fluid conduction and transfer system according to the invention observes mechanical compatibility with the Luer system, which closes or opens the connection of two parts by means of a half turn. As a result of the integration of a circuit (IC) 170 with a receiver coil 200, 202 in at least one first connecting piece 100, 101 of the Luer lock connection and a further coil 200, 202 in a second connecting piece 100, 101 of the Luer lock connection, both data and energy can be transmitted. Such connecting pieces are used in the infusion system 10 according to FIG. 1.

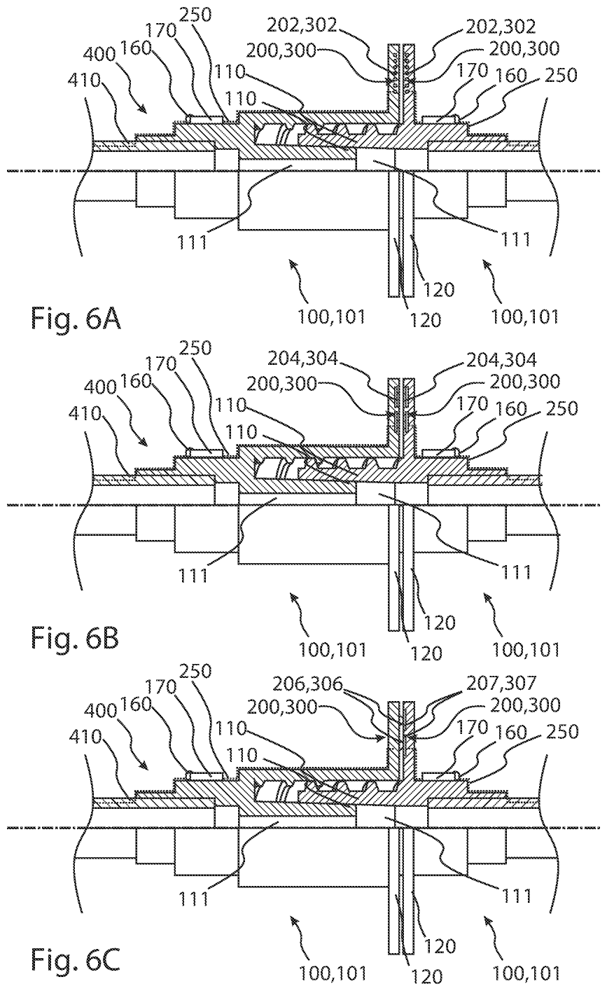

The connecting pieces of FIGS. 6A to 8C which follow are preferably also Luer lock connecting pieces according to ISO 80369-7. However, the connecting pieces can also be other connecting pieces according to the ISO 80369 family of standards.

FIGS. 6A to 6C show three versions of a connection of two connecting pieces 100, 101 having planar transmission devices 202, 204, 206, 207, 302, 304, 306, 307.

Regarding the essential features, the connecting pieces 100, 101 of FIG. 6A correspond to the design of FIG. 5. At the end of the two fluid lines 400 is, in each case, a connecting piece 100, 101, which is attached to the tube body 410 and has in each case a conduction section 110 through which a fluid channel 111 passes. The right connecting piece 100, 101 in FIGS. 6A to 6C is female and the left connecting piece 100, 101 is male. This means that the conduction section 110 of the left connecting piece 100, 101 is pushed into the conduction section 110 of the right connecting piece 100, 101. The conduction sections 110 are conical in order to create a secure fluid connection.

Besides the liquid communication through the conduction section 110, the connecting pieces 100, 101 are arranged in relation to one another in such a way after coupling that data transmission and/or energy transmission is possible owing to the coils 202, 302 in the region of collars 120 integral with the conduction sections 110, which coils were already described in relation to FIG. 5.

In the design according to FIG. 6B, the two connecting pieces each have two disk-shaped concentric electrode elements 204, 304 for capacitive coupling in a circumferential collar 120. As a result, bidirectional communication between the connecting pieces 100 is possible, for example.

In the design according to FIG. 6C, disk-shaped circumferential contact surfaces 207 are provided on the side of the right connecting piece 100, 101. Corresponding sliding contacts 206 are arranged on the left connecting piece.

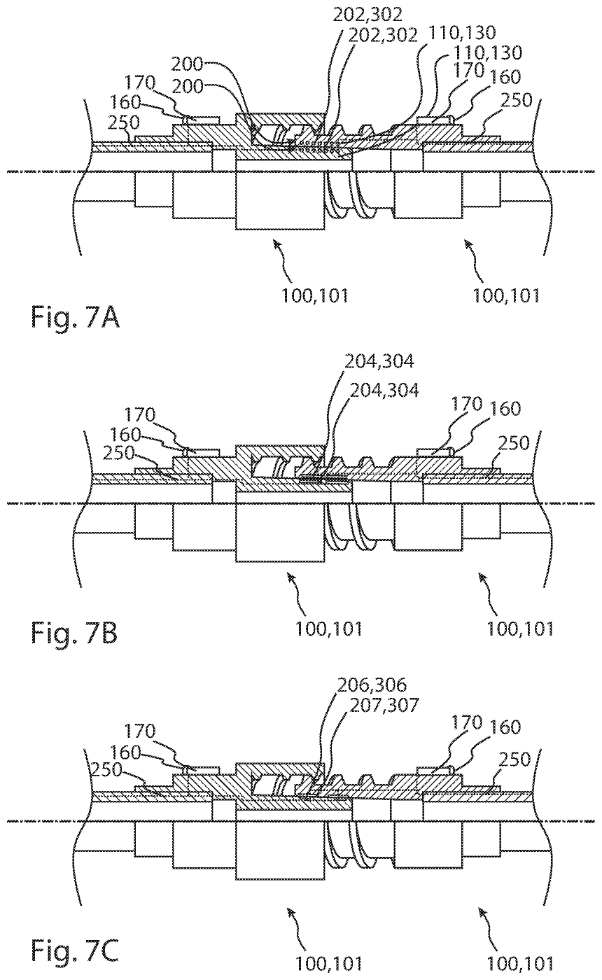

FIGS. 7A to 7C likewise show connecting pieces 100, 101 having conical conduction sections 110. However, here, the elements for data or energy transmission, i.e., the coils 202, 302, the contact surfaces 206, 306, 207, 307 or the electrode elements 204, 304, are not planar, but approximately annular or sleeve-shaped. They are integrated on or in the walls of the conduction sections 120. This means that reliable transmission is possible even with quite different insertion depths, as are possible with tapered connections. A further advantage resulting from the use of the conduction sections as carrier of the transmission devices is that rotatable coupling means such as union nuts can be used, the rotatability of which would make it structurally difficult to use a collar according to FIGS. 6A to 6C for the attachment of the transmission devices.

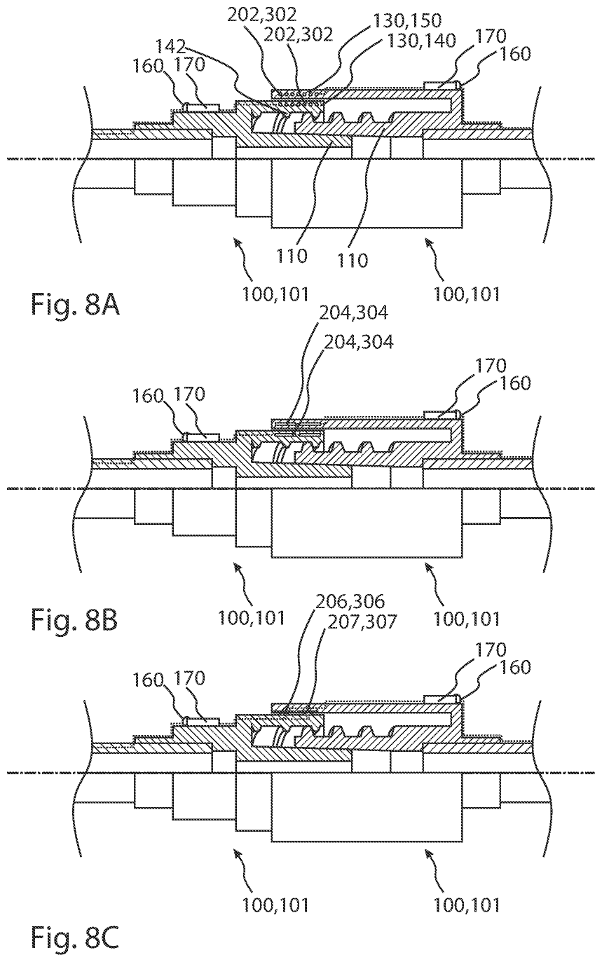

FIGS. 8A to 8C again show connecting pieces 100, 101 having coils, contact surfaces or electrode surfaces which are annular or sleeve-shaped. However, here, the corresponding elements are provided, firstly, in the region of an outer side of a coupling section 140 and, secondly, on a sheath wall 150 enveloping the coupling section.

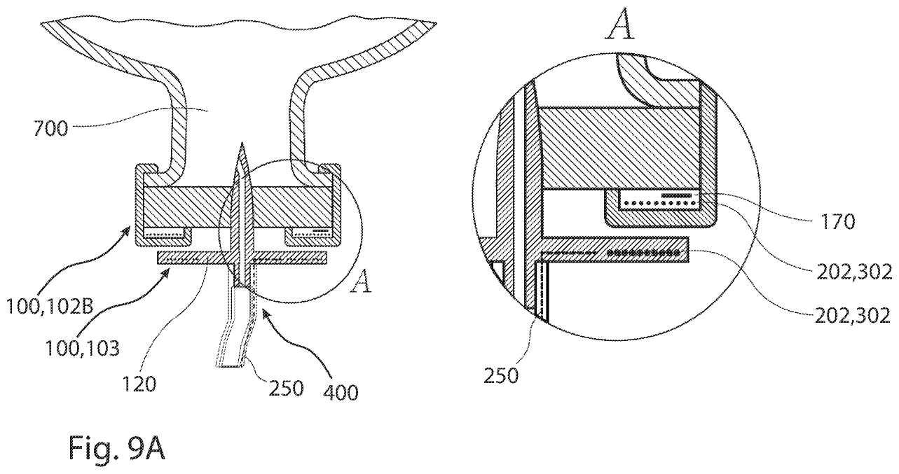

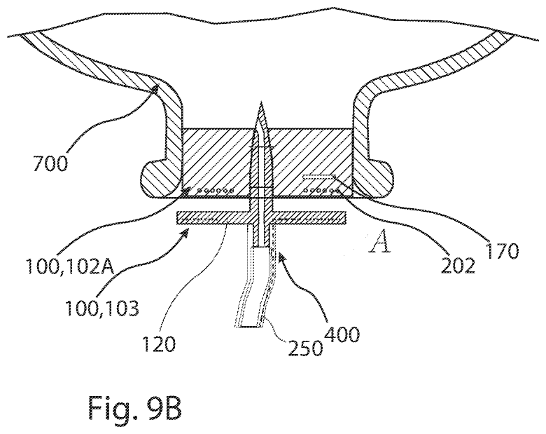

FIG. 9A shows a connection between an infusion bag 700 or an infusion bottle and an infusion tube 400 having expanded electronics. What is integrated in the infusion bag 700 or its connecting piece 100 formed by the crimp cap 100, 102B according to ISO 8536-3 is a circuit (IC) 170 with a receiver coil 202, and what is comprised in the infusion tube 400 to be attached is a connecting piece 103 according to ISO 8536-4 with a further coil 202, meaning that the two components—if they have been connected to one another—are inductively coupled with regard to information and energy. As a result, data and energy can be transmitted and exchanged via the integrated data line 250 in the infusion tube 400.

FIG. 9B shows an alternative design in which the connecting piece 100 on the sides of the infusion bag 700 or the infusion bottle 700 is a plug 100, 102A according to ISO 8536-2, equipped with a transmission coil 202 and a circuit 170.

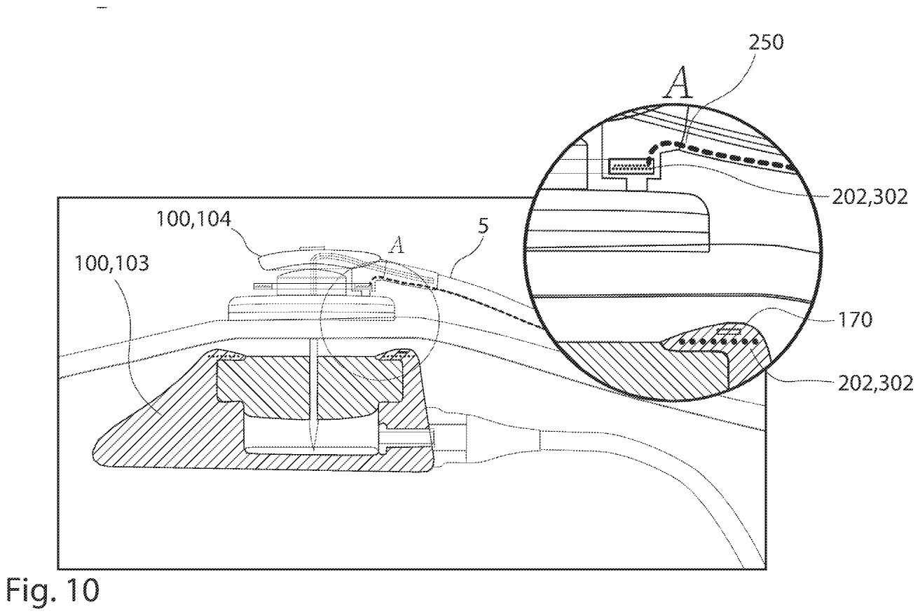

FIG. 10 shows the connection between connecting pieces 100 in the form of an implanted port 100, 103 and a port needle 100, 104 according to ISO 10555-6 and a cannula of the port needle with expanded electronics as an example of an access unit. What is situated in the implanted port 100, 103 is a circuit 170 with a receiver or transmission coil 202, 302, and what is integrated in the port needle 100, 104 is a further receiver or transmission coil 202, 302 which is connected to the data line 25, which is parallel to the infusion solution in the infusion tube (i.e., the connecting conductor system). As a result, the transmission of data and energy between the infusion system including the port needle 100, 104 and the implanted port 100, 103 is possible.

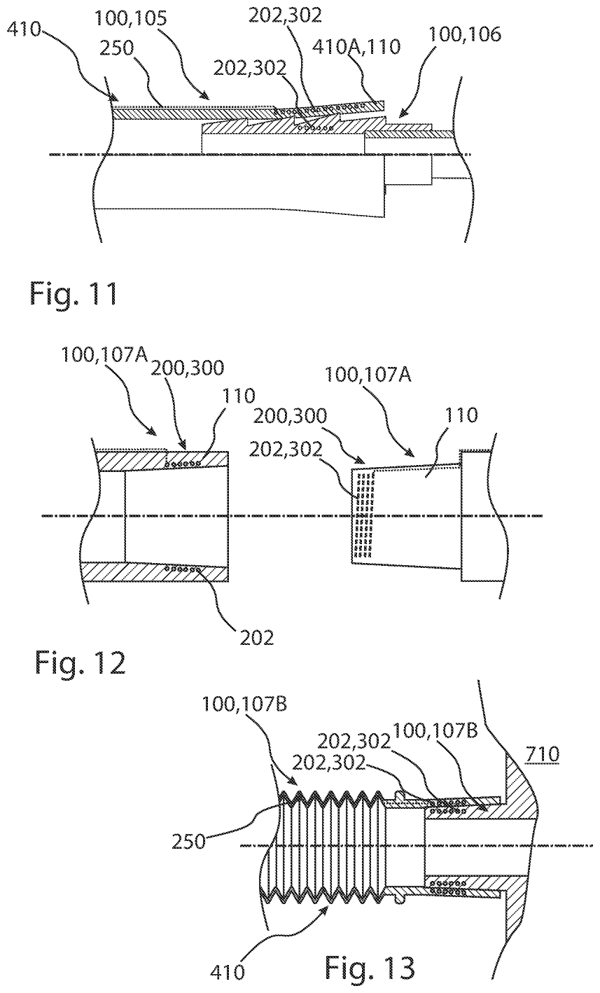

On the left-hand side, FIG. 11 shows a conduction body 410 of a fluid line 400 in the form of a hose body, the connecting piece 100 of which is directly part of the hose body. The transmission device of the connecting piece is formed by a sleeve-shaped coil 202 which is surrounded by the material of the hose body. This connecting piece 100 integrated in the hose body is provided on a counter-connecting piece 100, depicted on the right, in the form of a stepped clamp sleeve. This too is provided with a sleeve-shaped coil 202 which, in the coupled state depicted, is arranged opposite the coil 202 of the other connecting piece, so that high efficiency of energy transmission and reliable data transmission are achieved. Such connecting pieces 100 are used in the drainage system 14 according to ISO 20697 according to FIG. 3.

FIG. 12 shows two connecting pieces 100, 107A according to ISO 5356-1 for a ventilation system. Similar to depictions 7A, transmission devices 200 with coils 202 are also provided here, which are integrated in the conical conduction sections 110 of the connecting pieces.

FIG. 13 shows connectors 107B according to ISO 5367, which are likewise used in a ventilation system such as that in FIG. 4, especially for connection of a fluid line 400 having a hose body 410 to the ventilation machine 710. The connectors 107B are in turn each formed with a coil 202 for data and/or energy transmission. One of the connectors 107B or both connectors 107B can be additionally provided with an integrated circuit that is not depicted.

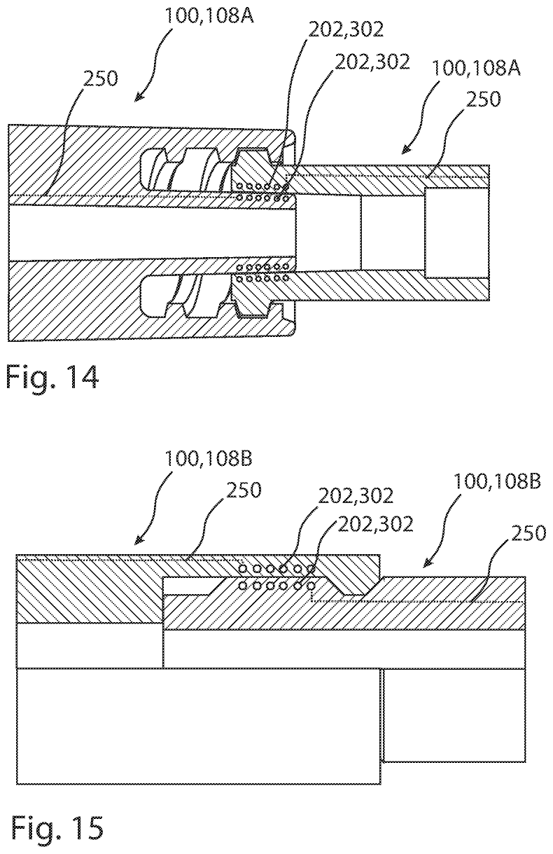

FIG. 14 shows two connecting pieces 108A of a blood port of a dialysis cartridge 600. The connecting pieces 108A are connecting pieces according to ISO 8637-1 and ISO 8637-2, which are designed for a tight connection by means of conical conduction sections. Again, conductors 250 are provided on the connecting pieces 108A, which are connected to coil 202 for data and/or energy transmission.

FIG. 15 shows two connecting pieces 108B according to ISO 8637-1 of a dialysate port of a dialysis cartridge 600. The connecting piece depicted on the right-hand side is usually provided on the dialysis cartridge 600. It has a circumferential groove into which a latching element of the connecting piece 108B on the left-hand side latches. Here too, conductors 250 are provided on the connecting pieces 108B, which are connected to coil 202 for data and/or energy transmission.

The connecting pieces 102A, 102B, 103, 104, 105, 106, 107A, 107B, 108A, 108B that have been shown and described can be provided with transmission devices of different types (capacitive, inductive, galvanic) in the manner elucidated in FIGS. 6A to 8C and can have the arrangements described there (planar or annular in the conduction section or in a coupling section).

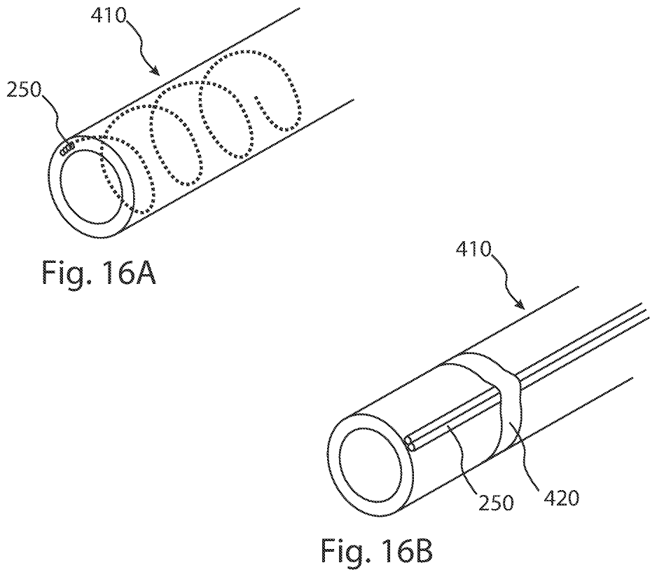

FIGS. 16A and 16B show different designs of the conduction body 410 of fluid lines 400. In each case, conductors 250 for transmission of electrical energy or for data transmission run parallel to the conduction body 410 of the fluid lines 400. In the design according to FIG. 16A, the conductors are embedded in the material of the conduction body. Furthermore, the conductors in this design are laid helically around the central fluid channel.

In the design according to FIG. 16B, the conductor 250 is arranged outside the conduction body 410, but connected thereto by circumferential coupling bands 420.

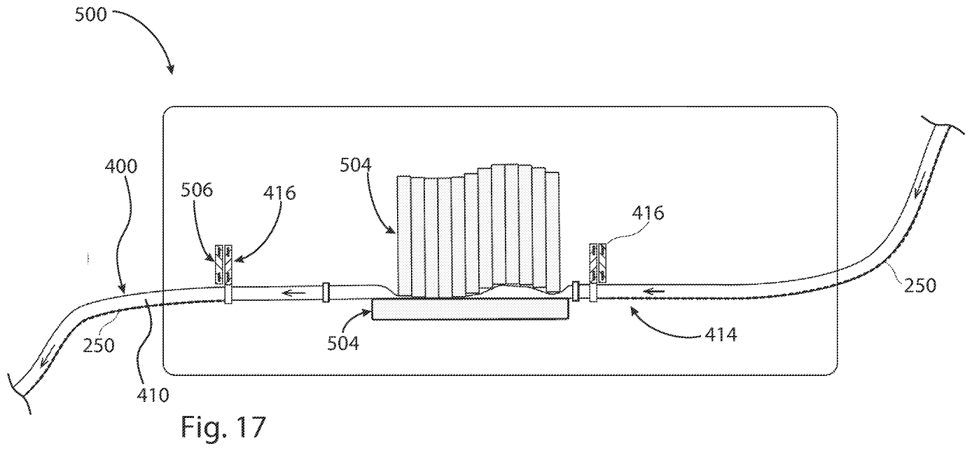

FIG. 17 shows an infusion pump 500, as can be used in the infusion system 10. The infusion pump 500 is designed as a peristaltic pump. It has a pressure device 502 comprising a plurality of plungers which are arranged above a counter-pressure surface 504. Provided between the pressure device 502 and the counter-pressure surface 504 is an accommodation space for a fluid line 400. If the fluid line 400 is inserted in the state of FIG. 17, fluid can be conveyed through the fluid line 400 by means of the pressure device and the plungers thereof.

The fluid line 400 is provided with two conductors 250, which are connected to connecting pieces, not depicted in FIG. 17, at the line ends upstream and downstream of the pressure device 502. No conductor is provided in the central region in which force is applied to the fluid line by the pressure device. Instead, the conductors 250 end at, in each case, a connecting device 416 in the form of a coil for inductive transmission of electrical power and data, which coil is laterally attached to the conduction body and is provided in the immediate vicinity of a coil 506 fixedly arranged in the conveying device 500.

In this way, the conveying device 500 or an integrated circuit of the conveying device can communicate with further components of the infusion system upstream and downstream or supply them with electrical power.

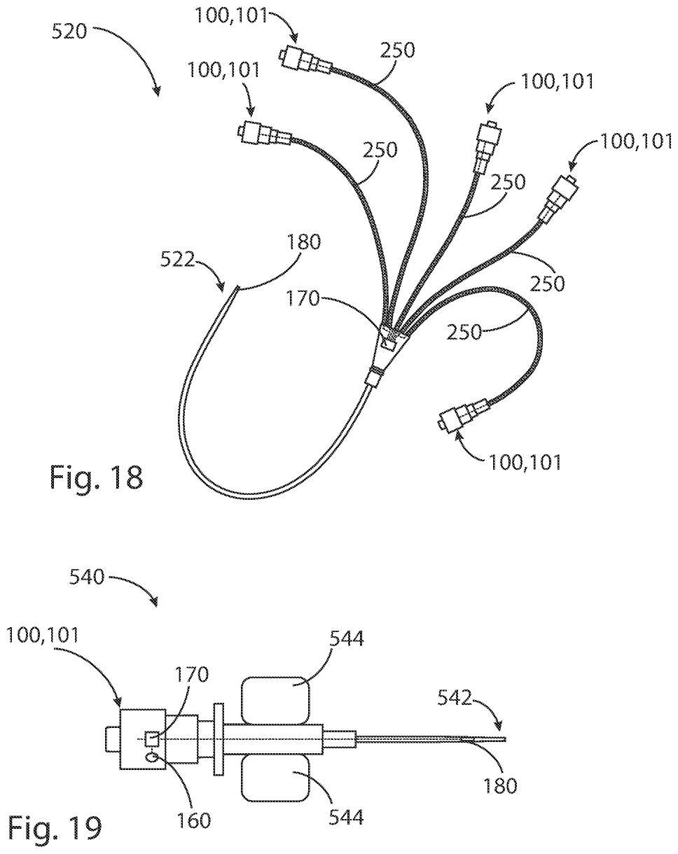

FIG. 18 shows a central venous catheter 520. It has an access tube 522 which is fed by five inputs. They are each provided with a connecting piece according to the invention. All the connecting pieces or elements of the infusion system 10 that are coupleable thereto communicate via conductors 250 with an integrated circuit 170. It can, on the basis of these data, bring together what fluid sources are connected to the connecting pieces 100. As a result, problematic fluid combinations or else deviations from a medication plan are detectable. Also possible is a design in which the integrated circuit 170 additionally communicates with a sensor 180 which acquires sensor data in the body of the patient.

The peripheral catheter 540 of FIG. 19 likewise has an access tube 542. However, it is only fed by one line designed as a connecting piece 100. Usually, the catheter is provided with wings 544 for attachment.

The transmission device in the connecting piece 100 is provided with an integrated circuit which, as in the case too of the catheter of FIG. 14, can communicate with a sensor 180. Moreover, an LED 160 is provided, which can, for example, indicate if a specified limit value is exceeded or fallen short of by the sensor data.

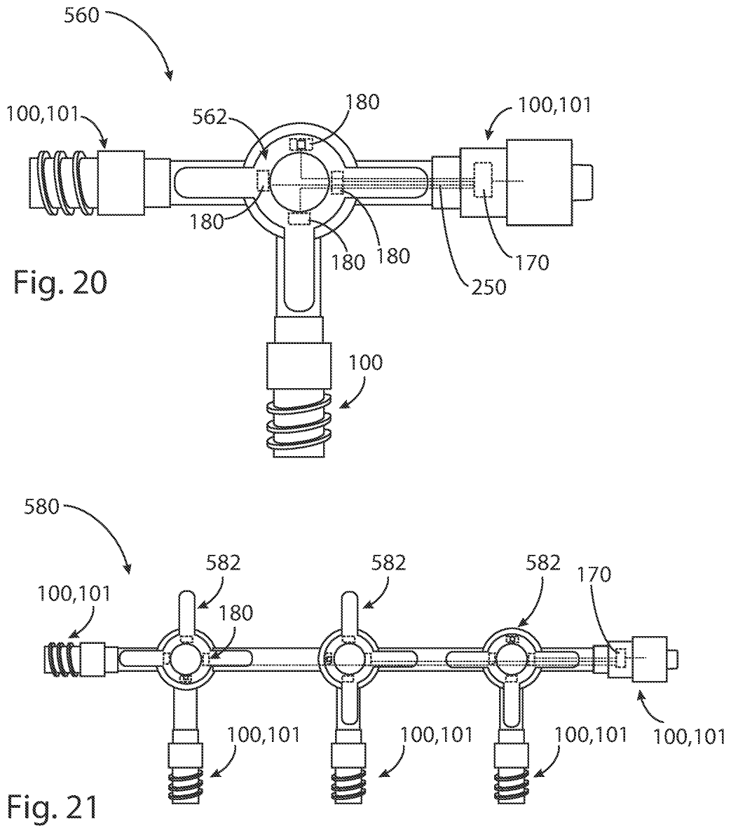

FIG. 20 shows a three-way valve which has three connecting pieces 100 altogether.

The three-way valve has, in a manner not depicted in detail, a directional regulating valve which can be adjusted manually by means of a handle 562 in order to control which input is connected to the output in a communicating manner.

To capture the current state of the directional regulating valve, sensors 180, for example Hall sensors, are provided, which capture the position of a magnet 182 attached to the valve or to the handle. This information can be transmitted from an integrated circuit 170 to a connected component, for example an integrated circuit of the conveying device 500 that acts as a master.

FIG. 21 shows a valve bank 580, the structure of which functionally corresponds to coupling of multiple three-way valves. The valve bank 580 has three directional regulating valves having handles 582, which, in the same manner as the three-way valve of FIG. 17, with sensors 180 for capture and transmission of the current switching state by means of an integrated circuit 170.

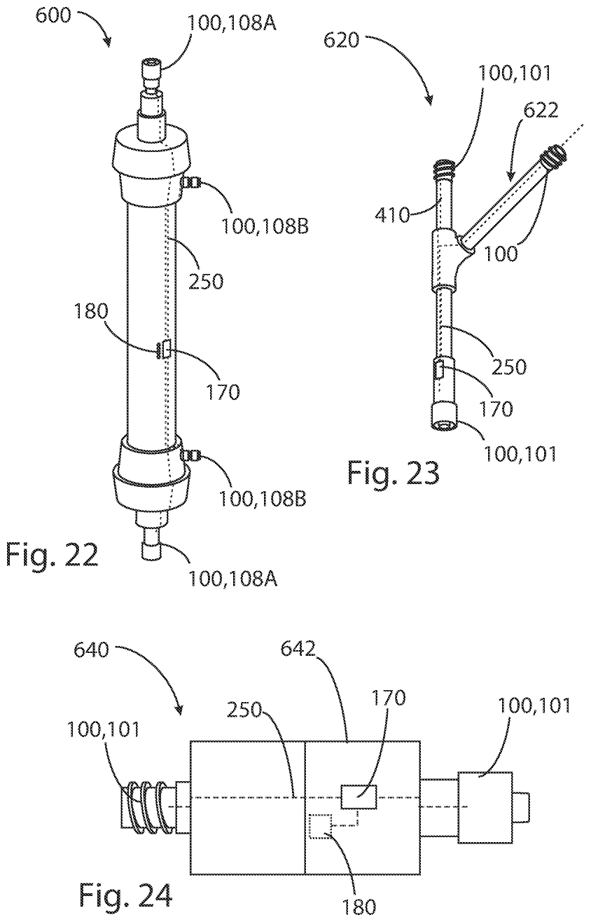

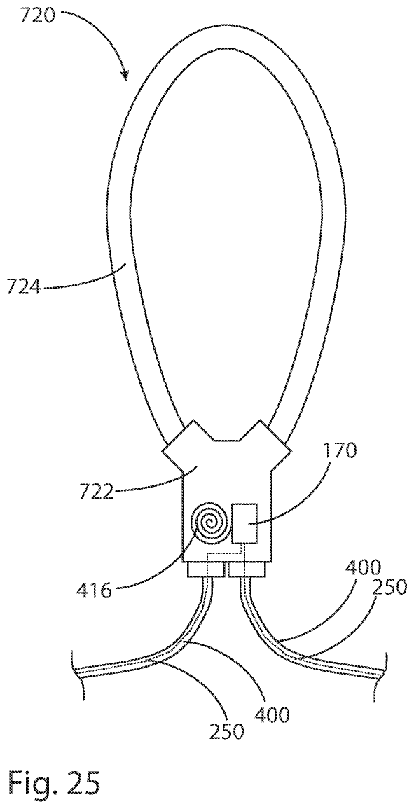

FIG. 22 shows a dialysis cartridge 600, as is used in the system 12 of FIG. 2. The dialysis cartridge 600 has a connecting piece 100 at the upper end, which is provided for the admittance of the blood, and a connecting piece 100 at the lower end, at which the blood is delivered. The lateral connecting pieces 100 form the dialysate inflow and the dialysate outflow.

All four connecting pieces 100 are designed as connecting pieces according to the invention having a data transmission device and are connected by means of conductors 250 to an integrated circuit 170 attached on the outside. It can communicate thereover with connected components in order to check the validity of the structure and/or to send relevant data for transmission to another validating integrated circuit.

FIG. 23 shows an auxiliary port device 620. It comprises two connecting pieces 100 at the upper end and at the lower end, which are connected by a tube line 410. In addition, an auxiliary port branch 622 is provided, which is preferably connected to the tube line 410 by means of a valve and on which an additional connecting piece is provided. An additional fluid source, for example a syringe, can be connected here ad hoc.

The three connecting pieces 100 altogether are each connecting pieces according to the invention. They are connected by means of conductors to an integrated circuit 170 which, for example, registers the connection of a syringe to the connecting piece 100 of the auxiliary port branch 622 and transmits this information onward.

FIG. 24 shows a filter unit 640 for filtration of fluid which flows through. The filter unit 640 has a filter housing 642 which has, at opposite ends, two connecting pieces 100 according to the invention as inlet and outlet. The connecting pieces are connected via conductors 250 to an integrated circuit. It is additionally also connected to a filter sensor 180, which captures filtration parameters and thereby allows the integrated circuit 170, in the event of an abnormality, to transmit the relevant information to a connected component.

FIG. 22 shows a tube unit 720 for a rotary peristaltic pump. Such a tube unit has a tube loop 724 which is placed around the rotor of the peristaltic pump. The two ends of the tube loop 724 are provided on a central element 722 which is connected to an inlet tube and an outlet tube 400, which preferably have at their ends connecting pieces 100 according to the invention, which are not depicted.

In addition to an integrated circuit 170 which controls communication via the connecting pieces, what is also provided on the central element is a connecting device 416 similar to that of FIG. 17, via which electrical power and/or data communication is made available for the tube unit 720 and associated components of the overall system.

1-53. (canceled)

54. A connecting piece for fluidic coupling of medical appliances and/or medical lines, comprising:
a connector of the ISO 80369 family of standards, a plug for an infusion bottle according to ISO 8536-2 comprising a crimp cap for an infusion bottle according to ISO 8536-3 or comprising a piercing spike according to ISO 8536-4 for connection to an infusion bottle or a bag, a subcutaneous implanted port according to ISO 10555-6, a non-coring cannula for coupling to an implanted port according to ISO 10555-6, a conical connector for anesthetic and respiratory equipment according to ISO 5356-1, a connector according to ISO 5367, a connecting piece for surgical wound drainage systems according to ISO 20697, a connecting piece of an extracorporeal circuit of a hemodialysis system, hemodiafiltration system or hemofilter system as a blood port according to ISO 8637-1, a connecting piece for connection to blood ports for dialysis systems according to ISO 8637-2, or a connecting piece for internal connection of a dialysis system as a port for dialysis liquid according to ISO 8637-1,
wherein the connecting piece:
is designed for coupling to a correspondingly designed counter-connecting piece;
has a fluid channel surrounded by a conduction section for circumferentially tight coupling in a plug-in direction to the counter-connecting piece and for subsequent exchange of liquids and/or gases with the counter-connecting piece; and
has a transmission device for data transmission and/or for transmission of electrical energy with the counter-connecting piece;
wherein the transmission device has at least one planar or helical coil surrounding the fluid channel.

55. The connecting piece as claimed in claim 54, wherein:
the at least one planar or helical coil comprises at least one planar coil, and the at least one planar coil is spiral shaped; or the at least one planar or helical coil comprises at least one helical coil, and the at least one helical coil is cylindrical or conical.

56. The connecting piece as claimed in claim 54, wherein:
the at least one planar or helical coil comprises at least one planar coil, the connecting piece has a circumferential collar, and the at least one planar coil is provided on or in the circumferential collar.

57. The connecting piece as claimed in claim 54, wherein:
the at least one planar or helical coil comprises at least one helical coil, the connecting piece has a sleeve-shaped ring section, on the inner side of which, on the outer side of which or within which the at least one helical coil is provided.

58. The connecting piece as claimed in claim 54, wherein the connecting piece:
is made from plastic at least in part, and/or
has at least one metallic or polymeric conductor for conducting electrical energy or for conducting data.

59. The connecting piece as claimed in claim 54, further including:
at least one output device.

60. The connecting piece as claimed in claim 54, further including:
an integrated circuit which is supplied with power by the transmission device for transmission of electrical energy and/or which is connected to the transmission device for data transmission.

61. The connecting piece as claimed in claim 54, further including:
a sensor for capturing a property of a fluid flowing through or for capturing a volumetric flow rate of the fluid flowing through, or for capturing a coupling state of the connecting piece on the counter-connecting piece.

62. A set of at least two connecting pieces, wherein:
each of the two connecting pieces comprises the connecting piece as claimed in claim 54 and are for mechanical coupling to one another;
the two connecting pieces being designed in such a way that, through mechanical coupling, respective fluid channels thereof are brought into communicating and circumferentially-tight connection with one another; and
the two connecting pieces being designed in such a way that, through mechanical coupling, respective transmission devices thereof are brought into a position relative to one another that allows data transmission or transmission of electrical energy.

63. A medical fluid line for transferring liquids and/or gases, comprising:
a conduction body comprising a rigid pipe body or a flexible tube body that surrounds a fluid channel; and
the connecting piece as claimed in claim 54 at a proximal end of the conduction body.

64. The medical fluid line as claimed in claim 63, further including:
a second connecting piece at a distal end of the fluid line; and/or
an electrically conductive conductor for conducting digital data and/or for conducting electrical energy along the conduction body; and/or
a wall of the conduction body within which the conductor is arranged.

65. A fluid-guiding medical appliance, wherein:
the appliance is designed for receiving, conducting and/or delivering fluids;
the appliance is designed for receiving, conducting and/or delivering data and/or electrical energy; and
the appliance has the connecting piece as claimed in claim 54 for combined receiving, conduction and/or delivery of data and/or electrical energy and of a fluid.

66. The fluid-guiding medical appliance as claimed in claim 65, further including:
at least one actuator for controlling the fluid and/or applying pressure thereto; and/or
at least one sensor for capturing a property of the fluid or for capturing a volumetric flow rate of the fluid; and/or
at least one integrated circuit which is supplied with power by the transmission device for transmission of electrical energy and/or is connected to the transmission device for data transmission.

67. The fluid-guiding medical appliance as claimed in claim 66, wherein:
the appliance is designed as a conveying unit.

68. The fluid-guiding medical appliance as claimed in claim 66, wherein:
the appliance is designed as a central venous catheter or as a peripheral venous catheter.

69. The fluid-guiding medical appliance as claimed in claim 66, wherein:
the appliance is designed as a valve bank or a multi-way valve.

70. The fluid-guiding medical appliance as claimed in claim 66, wherein:
the appliance is designed as a hemodialysis cartridge, a hemodiafilter cartridge, a hemofilter or a hemoconcentrator cartridge.

71. The fluid-guiding medical appliance as claimed in claim 66, wherein:
the appliance is designed as an auxiliary port device and has a fluid line provided with a connecting piece on both sides, between which there is provided an auxiliary port for ad hoc addition of a fluid.

72. The fluid-guiding medical appliance as claimed in claim 66, wherein:
the appliance is designed as a filter appliance.

73. The fluid-guiding medical appliance as claimed in claim 66, wherein:
the appliance is designed as a ventilation tube.

74. A medical fluid conduction and transfer system comprising:
an access unit for a patient for administrating a fluid or a fluid mixture into the patient and/or for withdrawing a fluid from the patient; and
at least one liquid container for providing or accommodating the fluid;
wherein the access unit and/or the liquid container has the connecting piece as claimed in claim 54.
